(19) United States
(12) Patent Application Publication
(10) Pub. No.: US 2011/0015150 A1
(43) Pub. Date: Jan. 20, 2011

(54) METHODS OF ADMINISTERING COMPOSITIONS INCLUDING IRON

(75) Inventors: Jonathan David Bortz, St. Louis, MO (US); Mitchell I. Kirschner, St. Louis, MO (US); David S. Hermelin, St. Louis, MO (US)

Correspondence Address: KV PHARMACEUTICAL COMPANY, One Corporate Woods Drive, BRIDGETON, MO, 63044, US

(73) Assignee: DRUGTECH CORPORATION, Wilmington, DE (US)

(21) Appl. No.: 12/891,376

(22) Filed: Sep. 27, 2010

Related U.S. Application Data
(63) Continuation of application No. 11/020,801, filed on Dec. 22, 2004, now abandoned.

(51) Int. Cl. A61K 31/714; A61K 31/341; A61K 31/519; A61P 7/06

(52) U.S. Cl. 514/52; 514/474; 514/249

(57) ABSTRACT

Nutritional or dietary supplement compositions that promote and/or maintain dietary iron absorption through administration of iron with an organic acid and optionally similar iron absorption promoters are provided. Also provided are methods of nutritional or dietary supplementation using one or more compositions that promote and/or maintain health through the prevention, stabilization, reversal and/or treatment of disorders associated with iron deficiency.

### FIELD OF THE INVENTION

The present invention relates to nutritional or dietary supplement compositions that promote dietary iron absorption through the administration of iron with one or more organic acids and optionally, similar iron absorption promoters. More specifically, the present invention relates to nutritional or dietary iron supplement compositions that include iron, one or more organic acids and optionally, similar iron absorption promoters, preferably used with cyclical administration to enhance dietary iron absorption so as to prevent, stabilize, reverse and/or treat disorders related to iron deficiency, such as iron deficiency anemia. Compositions of the present invention may be used independently to promote and/or maintain iron absorption or used in combination with one or more other compositions used in the treatment of one or more diseases having iron deficiency associated therewith.

### BACKGROUND OF THE INVENTION

Vitamin, multi-vitamin, and/or mineral preparations are commonly administered to inhibit, prevent, or reduce the frequency or severity of specific medical disorders. In particular, iron-containing preparations are used to alleviate disorders related to iron deficiency, such as for example iron deficiency anemia. Such vitamin, multi-vitamin, mineral and/or iron-containing preparations are also used as nutritional supplements.

Iron deficiency anemia is ubiquitous. In parts of Africa and Asia, where marginal dietary intake of iron and excessive iron loss owing to intestinal parasites occur together, more than 50 percent of the population may suffer from iron deficiency anemia. Iron-containing preparations have been available to treat iron deficiency anemia since the late 19^{th} century. Oral ferrous sulfate remains the conventional choice for dietary iron supplementation as it is considered a safe, cheap and effective means of replenishing iron stores in the vast majority of anemic patients. However, oral ferrous sulfate supplementation has considerable disadvantages associated with its use including such side effects as nausea, vomiting and constipation. Side effects of oral ferrous sulfate supplementation are due, at least in part, to the relatively large daily doses required to achieve adequate absorption and hemoglobin response.

Iron-containing preparations or “iron supplements”, optionally also containing other beneficial vitamins and/or minerals, are well known sources of dietary iron to treat or prevent iron deficiency in mammals. Commonly available iron supplements generally include a single form of iron. Examples of common single forms of iron used in iron supplements include iron (II) salt, i.e., a salt containing divalent or ferrous iron (III) salt, i.e., a salt containing trivalent or ferric iron and iron (O) powder, e.g., carbonyl iron.

Iron supplements are available commercially in rapid release dosage forms and in controlled release dosage forms. Rapid release iron supplement dosage forms typically contain a “rapidly dissolving” iron salt. Certain iron salts are significantly more soluble in water and gastrointestinal fluids than other salts and metallic forms of iron. Hence, these more soluble iron salts or “rapidly dissolving” iron salts are incorporated into rapid release iron supplement dosage forms. Administration of rapid release iron supplement dosage forms can cause excessively high maximum (max) blood-iron concentrations (C), i.e., Cₘₐₓ, within a short period of time (T) between administration and attainment of Cₘₐₓ, i.e., Tₘₐₓ. Accordingly, rapid release iron supplement formulations can cause unpleasant, harmful, or even fatal side effects. Such side effects may include stomach irritation, constipation, and iron poisoning.

Controlled release iron supplement dosage forms were developed in an attempt to reduce side effects such as those noted above, commonly associated with known iron supplementation therapies. Prior art controlled release iron supplement dosage forms commonly use an iron (II) salt encapsulated in or mixed with a release rate modifying matrix, an iron (Ill) salt, carbonyl iron or other metallic iron of naturally poor solubility, crystalline iron oxide, iron salt or carbonyl iron complexed with a release rate modifying protein, amino acid, organic acid, natural polymer, anionic complexing agent or synthetic polymer. Administration of such known controlled release iron supplement dosage forms generally results in temporary reductions of blood-iron concentrations between consecutive doses. Controlled release iron supplement dosage forms typically have a varying iron release rate, i.e., an initial relatively slow release rate, an intermediate relatively moderate release rate and a final relatively slow release rate. Temporary reductions of blood-iron concentrations can be due to the combined affects of a final relatively slow iron release rate from a first dose coupled with an initial relatively slow iron release rate from a second dose. Certain iron supplements designed to provide “sustained delivery” of iron, to avoid temporary reductions of blood-iron concentrations as noted above, have been associated with unpleasant tastes and odors, nausea, stomach irritation and gas formation.

It is clear that many options exist in the treatment of disorders associated with iron deficiency through the use of any one of a variety of iron supplement dosage forms. However, many such treatment options are associated with unpleasant or harmful side effects. There is therefore a need for a nutritional or dietary iron supplement that effectively prevents, stabilizes, reverses and/or treats disorders related to iron deficiency while minimizing or eliminating many, if not all, unpleasant or harmful side effects.

### SUMMARY OF THE INVENTION

The present invention relates to nutritional or dietary supplement compositions for administration to humans or other animals to prevent, stabilize, reverse and/or treat disorders associated with iron deficiency, such as for example iron deficiency anemia. The present nutritional or dietary supplement compositions preferably comprise an effective amount of one or more forms of iron, an effective amount of one or more organic acids such as for example but not limited to one or more forms of succinic acid, and optionally one or more similar iron absorption promoters. Health is promoted and/or maintained though use of the present compositions by increased iron absorption and reduced detrimental side effects. Compositions of the present invention may be used independently to promote and/or maintain iron absorption or used in combination with one or more other compositions used in the treatment of one or more diseases having iron deficiency associated therewith.

The present invention likewise provides a method of treating a human or other animal by administering a nutritional or dietary supplement composition comprising an effective amount of one or more forms of iron, an effective amount of one or more organic acids such as for example but not limited to one or more forms of succinic acid and optionally an effective amount one or more similar iron absorption promoters. The practice of this invention involves supplementing the diet of humans or other animals by enteral and/or parenteral administration such as but not limited to oral, intraperitoneal, intravenous, subcutaneous, transcutaneous or intramuscular routes of administration using one or more compositions of the present invention. Compositions of the present invention are preferably used with cyclical administration. “Cyclical administration” of the present compositions, as used herein, means administration of one or more of the subject compositions in one or more dosage forms, in one or more dosage units, one or more times a day on a regular basis with regular intermittent periods of non-iron administration. Regular intermittent periods of non-iron administration create decreases in small intestine mucosal cell iron pools. Decreases in small intestine mucosal cell iron pools increases or optimizes iron absorption, as is discussed in more detail below.

The present invention likewise provides a method of manufacturing nutritional or dietary supplement compositions comprising an effective amount of one or more forms of iron, an effective amount of one or more organic acids such as for example but not limited to one or more forms of succinic acid, and optionally an effective amount of one or more similar iron absorption promoters to treat disorders associated with iron deficiency.

Accordingly, it is an object of the present invention to provide nutritional or dietary supplement compositions effective in the prevention, stabilization, reversal and/or treatment of one or more disorders associated with iron deficiency.

Another object of the present invention is to provide safe nutritional or dietary supplement compositions for the prevention, stabilization, reversal and/or treatment of iron deficiency anemia.

Another object of the present invention is to provide an effective method of preventing, stabilizing, reversing and/or treating one or more disorders associated with iron deficiency.

Another object of the present invention is to provide a safe method of preventing, stabilizing, reversing and/or treating one or more disorders associated with iron deficiency.

Another object of the present invention is to provide a method of manufacturing safe nutritional or dietary supplement compositions for the prevention, stabilization, reversal and/or treatment of one or more disorders associated with iron deficiency.

Still another object of the present invention is to provide a method of manufacturing nutritional or dietary supplement compositions effective in the prevention, stabilization, reversal and/or treatment of one or more disorders associated with iron deficiency.

These and other objectives and advantages of the present invention, some of which are specifically described and others that are not, will become apparent from the detailed description and claims that follow.

### DETAILED DESCRIPTION OF THE INVENTION

The present invention relates to nutritional or dietary supplement compositions for administration to humans or other animals to prevent, stabilize, reverse and/or treat disorders associated with iron deficiency, such as for example iron deficiency anemia. The present nutritional or dietary supplement compositions preferably comprise an effective amount of one or more forms of iron, an effective amount of one or more organic acids such as for example but not limited to one or more forms of succinic acid, and optionally an effective amount of one or more similar iron absorption promoters.

The preferred form of iron in the present compositions is Ferrochel™ (Albion International, Inc., Clearfield, Utah) a commercially available bis-glycine chelate of iron. Ferrochel™ is the preferred form of iron for the present invention due to its gentleness to the stomach or tolerability profile. While the bis-glycine chelate of iron is preferred, any number of suitable chelates may be used. For example, amino acid chelates are becoming well accepted as a means of increasing the metal content in biological tissues of man, animals and plants. Amino acid chelates are products resulting from the reaction of a polypeptide, dipeptide or naturally occurring alpha amino acid with a metal ion having a valence of two or more. The alpha amino acid and metal ion form a ring structure wherein the positive electrical charges of the metal ion are neutralized by the electrons of the carboxylate or free amino groups of the alpha amino acid. Although the term amino acid as used herein refers only to products obtainable through protein hydrolysis, synthetically produced amino acids are not to be excluded provided they are the same as those obtained through protein hydrolysis. Accordingly, protein hydrolysates such as polypeptides, dipeptides and naturally occurring alpha amino acids are collectively referred to as amino acids. Additional suitable amino acid chelates include for example but are not limited to ethylenediaminetetraacetic acid (EDTA), monohydroxyethylethylenediaminetriacetic acid, diethylenetriaminepentaacetic acid, monohydroxyethyldiglycine and dihydroxyethylglycine.

Other suitable forms of iron for purposes of the present invention include for example but are not limited to soluble iron salts, slightly soluble iron salts, insoluble iron salts, chelated iron, iron complexes, non-reactive iron such as carbonyl iron and reduced iron, and combinations thereof.

Preferred chelated iron complexes are disclosed in U.S. Pat. Nos. 4,599,152 and 4,830,716, each incorporated herein by reference.

Examples of suitable soluble iron salts include but are not limited to ferric hypophosphite, ferric albuminate, ferric chloride, ferric citrate, ferric oxide saccharate, ferric ammonium citrate, ferrous chloride, ferrous gluconate, ferrous iodide, ferrous sulfate, ferrous lactate, ferrous fumarate, heme, ferric trisglycinate, ferrous bisglycinate, ferric nitrate, ferrous hydroxide saccharate, ferric sulfate, ferric gluconate, ferric aspartate, ferrous sulfate heptahydrate, ferrous phosphate, ferric ascorbate, ferrous formate, ferrous acetate, ferrous malate, ferrous glutamate, ferrous cholinisocitrate, ferroglycine sulfate, ferric oxide hydrate, ferric pyrophosphate soluble, ferric hydroxide saccharate, ferric manganese saccharate, ferric subsulfate, ferric ammonium sulfate, ferrous ammonium sulfate, ferric sesquichloride, ferric choline citrate, ferric manganese citrate, ferric quinine citrate, ferric sodium citrate, ferric sodium edetate, ferric formate, ferric ammonium oxalate, ferric potassium oxalate, ferric sodium oxalate, ferric peptonate, ferric manganese peptonate, other pharmaceutically acceptable iron salts, and combinations thereof.

Examples of suitable slightly soluble iron salts include but are not limited to ferric acetate, ferric fluoride, ferric phosphate, ferric pyrophosphate, ferrous pyrophosphate, ferrous carbonate saccharated, ferrous carbonate mass, ferrous succinate, ferrous citrate, ferrous tartrate, ferric fumarate, ferric succinate, ferrous hydroxide, ferrous nitrate, ferrous carbonate, ferric sodium pyrophosphate, ferric tartrate, ferric potassium tartrate, ferric subcarbonate, ferric glycerophosphate, ferric saccharate, ferric hydroxide saccharate, ferric manganese saccharate, ferrous ammonium sulfate, other pharmaceutically acceptable iron salts, and combinations thereof.

Examples of suitable insoluble iron salts include but are not limited to ferric sodium pyrophosphate, ferrous carbonate, ferric hydroxide, ferrous oxide, ferric oxyhydroxide, ferrous oxalate, other pharmaceutically acceptable iron salts and combinations thereof.

Examples of suitable iron complexes include but are not limited to polysaccharide-iron complex, methylidine-iron complex, ethylenediaminetetraacetic acid (EDTA)-iron complex, phenanthrolene iron complex, p-toluidine iron complex, ferrous saccharate complex, ferrlecit, ferrous gluconate complex, ferrum vitis, ferrous hydroxide saccharate complex, iron-arene sandwich complexes, acetylacetone iron complex salt, iron-dextran complex, iron-dextrin complex, iron-sorbitol-citric acid complex, saccharated iron oxide, ferrous fumarate complex, iron porphyrin complex, iron phtalocyamine complex, iron cyclam complex, dithiocarboxy-iron complex, desferrioxamine-iron complex, bleomycin-iron complex, ferrozine-iron complex, iron perhaloporphyrin complex, alkylenediamine-N,N-disuccinic acid iron(III) complex, hydroxypyridone-iron(III) complex, aminoglycoside-iron complex, transferrin-iron complex, iron thiocyanate complex, iron complex cyanides, porphyrinato iron(III) complex, polyaminopolycarbonate iron complexes, dithiocarbamate iron complex, adriamycin iron complex, anthracycline-iron complex, N-methyl-D-glucamine dithiocarbamate (MGD)-iron complex, ferrioxamine B, ferrous citrate complex, ferrous sulfate complex, ferric gluconate complex, ferrous succinate complex, polyglucopyranosyl iron complex, polyaminodisuccinic acid iron complex, biliverdin-iron complex, deferiprone iron complex, ferric oxyhydride-dextran complex, dinitrosyl dithiolato iron complex, iron lactoferrin complexes, 1,3-ethylenediaminetetraacetic acid (EDTA) ferric complex salts, diethylenetriaminepentaacetic acid iron complex salts, cyclohexanediaminetetraacetic acid iron complex salts, methyliminodiacetic acid iron complex salts, glycol ether diaminetetraacetic acid iron complex salts, ferric hydroxypyrone complexes, ferric succinate complex, ferric chloride complex, ferric glycine sulfate complex, ferric aspartate complex, sodium ferrous gluconate complex, ferrous hydroxide polymaltose complex, other pharmaceutically acceptable iron complexes and combinations thereof.

Suitable forms of iron for purposes of the present invention also include iron compounds designated as “slow dissolving” or “slow acting” and iron compounds designated as “fast dissolving” or “ fast acting”. Compositions of the present invention may optionally include at least two iron compounds, e.g., at least one iron compound designated slow acting and at least one iron compound designated as fast acting. The use of two such differing iron compounds in a formulation is disclosed in U.S. Pat. No. 6,521,247, incorporated herein in its entirety by reference. Compositions of the present invention may also include extended release iron compounds and/or controlled release iron compounds.

Compositions of the present invention include one or more forms of iron in an effective amount of about 10 mg to about 500 mg, more preferably about 50 mg to about 500 mg and most preferably from about 150 mg to about 500 mg per dosage. In the case of products developed for pediatric use, an effective amount of iron would be greatly reduced to levels considered safe for infants and children. An effective amount of one or more forms of iron for pediatric applications may be as low as about 0.5 mg of iron per kilogram of body weight per dosage.

Examples of suitable organic acids include but are not limited to succinic acid, acetic acid, citric acid, lactic acid, malic acid, glutamic acid and combinations thereof. Succinic acid is the preferred organic acid. Differing forms of such organic acids are also useful in compositions of the present invention. For example, not intended to be limiting, suitable forms of organic acids include for example but are not limited to succinic acid, acetic acid, citric acid, lactic acid, malic acid, glutamic acid, salts of succinic acid, salts of acetic acid, salts of citric acid, salts of lactic acid, salts of malic acid, salts of glutamic acid, derivatives of succinic acid, derivatives of acetic acid, derivatives of citric acid, derivatives of lactic acid, derivatives of malic acid, derivatives of glutamic acid and combinations thereof. Succinic acid, salts of succinic acid and derivatives of succinic acid are iron absorption promoters as described in still greater detail below. Compositions of the present invention include one or more forms of an organic acid or combinations thereof in an effective amount of about 5 mg to about 500 mg, more preferably about 100 mg to about 500 mg and most preferably about 150 mg to about 500 mg per dosage, to promote iron absorption. In the case of products developed for pediatric use, an effective amount of one or more forms of an organic acid or combinations thereof would be greatly reduced to levels considered safe for infants and children. An effective amount of one or more forms of an organic acid or combinations thereof for pediatric applications may be as low as about 0.50 mg of organic acid per kilogram of body weight per dosage.

Other suitable iron absorption promoters include for example but are not limited to ascorbic acid, salts of ascorbic acid, derivatives of ascorbic acid, compounds having Vitamin C activity, carbohydrates such as but not limited to mannitol, sorbitol, xylose, inositol, fructose, sucrose, lactose, and glucose, calcium, copper, sodium molybdate, amino acids and combinations thereof. “Compounds having Vitamin C activity” means Vitamin C (L-ascorbic acid) and any derivative thereof that exhibits ascorbic activity as determined by the standard iodine titration test. Derivatives of ascorbic acid include, for example, oxidation products such as dehydroascorbic acid and edible salts of ascorbic acid such as for example but not limited to calcium ascorbate, sodium ascorbate, magnesium ascorbate, potassium ascorbate and zinc ascorbate. Metabolites of ascorbic acid and its derivatives include for example but are not limited to aldo-lactones and edible salts of aldonic acids. Compositions of the present invention preferably include one or more ascorbic acid metabolites, namely, L-threonic acid, L-xylonic acid and L-lyxonic acid. A preferred form of ascorbic acid for purposes of the present invention is Ester C® (Zila Nutraceuticals, Inc., Prescott, Ariz.), as disclosed in U.S. Pat. Nos. 4,822,816 and 5,070,085, each incorporated herein by reference. Ester C® is a preferred form of ascorbic acid due to its enhanced health benefits. Compositions of the present invention optionally include one or more iron absorption promoters in addition one or more forms of an organic acid, in an effective amount of about 5 mg to about 500 mg, more preferably about 100 mg to about 500 mg and most preferably about 150 mg to about 500 mg per dosage, to promote iron absorption as discussed in still greater detail below.

Optionally, one or more of the individual components of compositions of the present invention may be formulated as coated or treated beads for controlled release to optimize absorption. In coating or treating the components, components could be coated or treated with the same coating or treatment, or could be coated individually with one or more differing coatings or treatments. Likewise one or more components could be coated or treated and combined with one or more components that are uncoated or untreated. Such coating or treatment variations are useful to manipulate and control the release of each component so as to optimize absorption. Such coating of components is described in more detail below in Example 16.

An example of a nutritional or dietary supplement composition of the present invention includes about 10 mg to about 500 mg of one or more forms of iron, about 25 mg to about 500 mg of one or more forms of succinic acid and about 25 mg to about 500 mg of one or more forms of ascorbic acid per dosage.

Another example of a nutritional or dietary supplement composition of the present invention includes about 10 mg to about 500 mg of carbonyl iron, chelated iron or mixtures thereof, about 25 mg to about 500 mg of succinic acid and about 25 mg to about 500 mg of ascorbic acid per dosage.

Another example of a nutritional or dietary supplement composition of the present invention includes about 10 mg to about 500 mg of one or more non-reactive iron compounds, about 25 mg to about 500 mg of succinic acid and about 25 mg to about 500 mg of ascorbic acid per dosage.

Another example of a nutritional or dietary supplement composition of the present invention includes about 151 mg elemental iron such as for example in the form of about 70 mg Ferrochel™ iron and about 81 mg ferrous fumarate iron, about 150 mg of one or more forms of succinic acid and about 200 mg of one or more forms of ascorbic acid per dosage.

Another example of a nutritional or dietary supplement composition of the present invention includes about 151 mg of one or more forms of elemental iron such as for example in the form of about 100 mg Ferrochel™ iron and about 50 mg ferrous fumarate iron, about 150 mg of one or more forms of succinic acid, about 60 mg of one or more forms of ascorbic acid, about 1.0 mg folic acid and about 10 mcg Vitamin B₁₂ per dosage.

Another example of a nutritional or dietary supplement composition of the present invention includes about 151 mg of one or more forms of elemental iron, about 150 mg of one or more forms of succinic acid, about 200 mg of one or more forms of ascorbic acid, about 1.0 mg folic acid and about 10 mcg Vitamin B₁₂ per dosage.

Still another example of a nutritional or dietary supplement composition of the present invention includes about 175 mg of one or more forms of elemental iron, about 150 mg of one or more forms of succinic acid, about 200 mg of one or more forms of ascorbic acid, about 1.0 mg folic acid and about 10 mcg Vitamin B₁₂ per dosage. Additionally, compositions of the present invention may be administered in combination with group B Vitamins, and/or laxatives, and/or anti-emetic agents, and/or birth control agents, and/or one or more other compositions used in the treatment of one or more diseases having iron deficiency associated therewith.

A dosage of one or more compositions of the present invention may be manufactured in one or more dosage forms such as for example but not limited to a tablet, caplet, capsule, gel capsule, chew tablet, lozenge and troche, nutritional bar or food item, soft chew, reconstitutable powder or shake, sprinkle, semi-solid sachet or the like. Any tablet dosage form may be either chewable or compressed. The preferred solid dosage form for purposes of the present invention is a capsule or tablet. However, compositions of the present invention could likewise be incorporated into a food product or a powder for mixing with a liquid. Although any number of suitable dosage forms can be used to administer compositions of the present invention, preferred dosage forms include a single capsule, two capsules or one capsule and one caplet or tablet. Compositions of the present invention can not only be provided in various dosage forms but can also be administered in accordance with various dosage regimens as described in more detail below. For example, a dosage of one or more compositions of the present invention may be administered as one more dosage units and in one or more dosage forms.

Compositions of the present invention are described in still more detail in the examples provided below. Such examples are provided for illustrative purposes only and are not intended to be limiting to the scope of the present invention.

### EXAMPLE 1

### Method of Making Composition of the Present Invention

Purified water (1.53 kg) was loaded into a stainless steel tank equipped with a mixer. While mixing, povidone (11.5 kg) was added to the purified water and mixed until all the solids were dissolved into solution. A fluid bed granulator dryer was then loaded with the ingredients amino acid chelated iron (162 kg), ferrous fumarate iron (54.9 kg), succinic acid (48.5 kg) and lactose monohydrate (79.7 kg). The ingredients were then dry mixed with an inlet temperature setting of approximately 70° C. to 90° C. until the exhaust temperature was approximately 54° C.±4° C. When the exhaust temperature reached approximately 54° C.±4° C., the ingredients were granulated using the solution prepared above. After granulation, the ingredients were dried until the exhaust temperature reached 60° C. to 70° C. The inlet temperature was then set to 25° C. until the exhaust temperature was below 45° C. The dried granulated ingredients were then milled and/or sized. The final material is loaded into double poly-lined containers for weight recording.

### EXAMPLE 2

### Composition Dosage of the Present Invention

A supplement composition was prepared in accordance with Example 1 containing 70 mg Ferrochel™ iron, 81 mg ferrous fumarate iron, 150 mg succinic acid, 200 mg ascorbic acid, 1.0 mg folic acid and 10 mcg Vitamin B₁₂.

### EXAMPLE 3

### Composition Dosage of the Present Invention

A supplement composition was prepared according to Example 1 containing 70 mg Ferrochel™ iron, 81 mg ferrous fumarate iron, 150 mg succinic acid, 1.0 mg folic acid and 10 mcg Vitamin B₁₂.

### EXAMPLE 4

### Composition Dosage of the Present Invention

A supplement composition was prepared according to Example 1 containing 70 mg Ferrochel™ iron and 150 mg succinic acid.

### EXAMPLE 5

### Composition Dosage of the Present Invention

A supplement composition is prepared according to Example 1 containing 25 mg Ferrochel™ iron and 60 mg succinic acid.

### EXAMPLE 6

### Composition Dosage of the Present Invention

A supplement composition is prepared according to Example 1 containing 25 mg Ferrochel™ iron, 60 mg succinic acid and 60 mg Vitamin C.

### EXAMPLE 7

### Composition Dosage of the Present Invention

A supplement composition is prepared according to Example 1 containing 150 mg Ferrochel™ iron, 150 mg succinic acid and 200 mg Vitamin C.

### EXAMPLE 8

### Composition Dosage of the Present Invention

A supplement composition is prepared according to Example 1 containing 150 mg Ferrochel™ iron and 150 mg succinic acid.

### EXAMPLE 9

### Composition Dosage of the Present Invention

A supplement composition is prepared according to Example 1 containing 100 mg Ferrochel™ iron, 50 mg ferrous fumarate iron, 150 mg succinic acid, 60 mg Vitamin C, 1.0 mg folic acid and 10 mcg Vitamin B₁₂.

### EXAMPLE 10

### Composition Dosage of the Present Invention

A supplement composition is prepared according to Example 1 containing 70 mg carbonyl iron, 81 mg ferrous sulfate iron, 150 mg malic acid, 1.0 mg folic acid and 10 mcg Vitamin B₁₂.

### EXAMPLE 11

### Composition Dosage of the Present Invention

A supplement composition is prepared according to Example 1 containing 70 mg ferrous fumarate iron complex, 81 mg ferrous sulfate iron, 150 mg lactic acid, 1.0 mg folic acid and 10 mcg Vitamin B₁₂.

### EXAMPLE 12

### Composition Dosage of the Present Invention

A supplement composition for pediatric use is prepared according to Example 1 containing 0.50 mg iron per kilogram of body weight of infant/child and 0.50 mg succinic acid per kilogram of body weight of infant/child.

### EXAMPLE 13

### Composition Dosage of the Present Invention

A supplement composition is prepared according to Example 1 containing 50 mg ferrous gluconate iron complex, 50 mg ferric phosphate iron, 50 mg ferric fumarate iron, 150 mg malic acid, 1.0 mg folic acid and 10 mcg Vitamin B₁₂.

### EXAMPLE 14

### Composition Dosage of the Present Invention

A supplement composition is prepared according to Example 1 containing 100 mg carbonyl iron, 100 mg ferrous sulfate iron, 250 mg lactic acid, 1.0 mg folic acid and 10 mcg Vitamin B₁₂.

### EXAMPLE 15

### Composition Dosage of the Present Invention

A supplement composition is prepared according to Example 1 containing 150 mg carbonyl iron, 100 mg ferrous fumarate iron, 200 mg citric acid, 1.0 mg folic acid and 10 mcg Vitamin B₁₂.

### EXAMPLE 16

### Polysaccharide Iron Complex Coated Beads

Polysaccharide iron complex, 7.0 kg, cellulose microcrystalline Avicel™ PH 101 (FMC, Brussels), 7.33 kg, colloidal silicon dioxide, NF, 0.15 kg and povidone, 0.53 kg, are added to a high shear granulator. The ingredients are mixed until uniformly blended. About 10 kg purified water is added while mixing the ingredients until granulation is complete. The wet granulation is then placed in an extruder with a screen. The wet granulation is extruded onto a tray and transferred to a spheronizer and spheronized. The wet sheronized pellets are then dried in an oven prior to passing the same through a screen to remove fines and oversized beads.

With mixing, 0.56 kg of a plasticizer such as diethyl phthalate, trie or triacetin, is slowly added to 7.5 kg of cellulose acetate phthalate aqueous dispersion Aquacoat™ CPD (Signet Chemical Corporation, Worli, Mumbai, India) and mixed for thirty minutes. Purified water, 10.69 kg, is then added to the ingredients and mixed for an additional 10 minutes. This Aquacoat DPD dispersion is then sprayed onto the sheronized polysaccharide iron complex pellets prepared above with fluidizing of the pellets, until the weight gain is 10 to 15 percent. The coated pellets or beads are then dried and cooled before placing in polylined containers.

Up until now, nutritional or dietary iron supplements have been formulated based on the premise that iron absorbed by an iron deficient human or animal would first go to red blood cell (RBC) production and only after RBCs reached normal levels would any absorbed iron go to iron stores within the body. This premise led to the administration of traditional iron compounds for about three months to supply the necessary amount of iron for RBCs assuming a 3.0 g hemoglobin deficit, and an additional six months to supply the necessary iron to replenish iron stores. Three months of iron supplementation for RBC regeneration is based on the fact that 150 mg of absorbed elemental iron is needed to produce 1.0 g of hemoglobin, and a 3.0 g rise in hemoglobin is a standard goal of iron therapy.

However, it has been found that during the first twenty days of nutritional or dietary iron supplementation, about 72 percent of the absorbed iron goes to RBC regeneration and about 28 percent of the absorbed iron goes to replenish iron stores within the body. For the next ten days of nutritional or dietary iron supplementation, i.e., days 21 through 30, 61 percent of the absorbed iron goes to RBC regeneration and 39 percent of the absorbed iron goes to replenish iron stores within the body. Therefore, during the important first 20 days of treatment, iron stores start to be replenished immediately. Accordingly, if iron absorption rates can be sufficiently increased, the duration of nutritional or dietary iron supplementation treatment therapies can be greatly reduced.

Based on generally accepted iron absorption rates, daily administration of ferrous sulfate containing 50 mg of elemental iron could require approximately 9 months to supply 450 mg of iron for RBC regeneration and 300 mg of iron to replenish iron stores within the body.

In a preferred embodiment of the present invention, an iron supplement composition dosage containing about 50 mg of Ferrochel™ iron, about 150 mg succinic acid and about 200 mg ascorbic acid administered once daily would require approximately 60 to 90 days to supply 450 mg of iron for RBC regeneration and 300 mg of iron to replenish iron stores within the body. Even more importantly, by administering daily an iron supplement composition of the present invention containing 150 mg of Ferrochel™ iron, 150 mg succinic acid and 200 mg ascorbic acid, would require approximately 20 to 40 days to supply 450 mg of iron for RBC regeneration and 300 mg of iron to replenish iron stores within the body.

A preferred nutritional or dietary iron supplement composition of the present invention comprises about 10 mg to about 500 mg of elemental iron, about 25 mg to about 500 mg of succinic acid and about 25 mg to about 500 mg of ascorbic acid per dosage. A dosage of such a composition may be administered once a day or more than once per day such as for example but not limited to morning administration and evening administration. Humans or other animals may be treated with compositions of the present invention using continuous administration or varying administration over the course of treatment. “Continuous administration” is the administration of a single composition formulation throughout the course of treatment. “Varying administration” is the administration of different composition formulations on different days, and/or administration of different composition formulations within a 24-hour period.

Suitable administration schedules or dosing regimens for purposes of the present invention also include administering one or more compositions of the present invention for about twenty-one days and then discontinuing iron supplementation for about seven days prior to again initiating iron supplementation. Such a dosing regimen is referred to herein as “cyclical administration”. Alternatively, one or more compositions of the present invention may be administered for about twenty days with discontinued iron supplementation for about 10 days, administered for about a week with discontinued iron supplementation for about a week, and the like. It is important to note that the present invention is not intended to be limited to administering one or more of the subject compositions for a specific number of days and then discontinuing iron supplementation for a specific number of days. Rather, iron supplementation is administered and discontinued for an amount of time necessary to affect a decrease in a labile pool of iron in small intestine mucosal cells. By affecting a decrease in the labile pool of iron in the small intestine mucosal cells, the potential for iron absorption by the small intestine mucosal cells is increased. During periods of discontinued iron supplementation, nothing, placebo, a non-iron containing composition comprising iron absorption promoters, vitamins, and/or minerals, one or more compositions useful in the treatment of one or more diseases associated with iron deficiency, or a combination thereof, may be administered.

In a preferred embodiment of the present invention, a nutritional or dietary iron supplement composition is provided for blood-iron concentration maintenance purposes. An illustrative composition for such blood-iron concentration maintenance includes 25 mg iron, 60 mg succinic acid and 100 mg ascorbic acid per dosage. Compositions for blood-iron concentration maintenance are useful for humans or other animals that are mildly iron deficient, post iron therapy, or are part of an “at risk” population, such as for example but not limited to regular blood donors.

As noted above, compositions of the present invention may be used independently to promote and/or maintain iron absorption, or used in combination with one or more other compositions used in the treatment of one or more diseases or conditions associated with iron deficiency. Such diseases or conditions associated with iron deficiency include for example but are not limited to gastro-intestinal diseases or conditions that cause blood loss such as for example but not limited to infectious parasites such as hookworms, regular use of non-steroidal anti-inflammatory drugs, steroids and/or aspirin, peptic ulcer disease, gastritis, colon cancer, polyps and inflammatory bowel disease, gastro-intestinal diseases or conditions that cause decreased absorption of iron such as for example but not limited to tropical sprue, celiac disease, autoimmune diseases, gastrectomy, gastric bypass, vagotomy and diseases requiring therapy with proton pump inhibitors and H2 antagonists, neurological diseases or conditions such as for example but not limited to restless leg syndrome, chronic fatigue, cognitive deficiencies and neuro-developmental deficiencies, physiological conditions such as for example but not limited to sports, menses, lactation, pregnancy and surgery, infectious diseases such as for example but not limited to HIV/AIDS and malaria, chronic diseases such as for example but not limited to cancer, rheumatoid arthritis and chronic renal failure and heavy metal poisoning such as for example but not limited to lead, mercury, cadmium and arsenic.

Nutritional or dietary iron supplement compositions of the present invention may also be provided for therapeutic purposes. An illustrative composition for therapeutic iron supplementation comprises 70 mg Ferrochel™ iron, 150 mg succinic acid and 200 mg ascorbic acid per dosage. This therapeutic nutritional or dietary supplement composition is useful for iron deficient humans or other animals. Such therapeutic compositions are preferably supplied in a once daily, 21-day calendar pack for monthly iron supplementation therapy. In such a case, absorbed iron provides sufficient iron for approximately 1.0 g per month of hemoglobin regeneration as well as iron for iron store repletion. It is preferable that iron supplementation be discontinued for at least a week following administration of the 21-day pack to allow absorption rates to remain high during administration weeks, thus optimizing the same. However, for women in their childbearing years, compositions of the present invention may be administered for seven days during menstruation to replenish lost iron, followed by discontinued iron supplementation for 21 days.

In yet another preferred embodiment of the present invention, a nutritional or dietary iron supplement composition is provided for therapeutic purposes. An illustrative composition for therapeutic iron supplementation includes 150 mg Ferrochel™ iron, 150 mg succinic acid and 200 mg ascorbic acid per dosage. This therapeutic nutritional or dietary supplement composition is useful for iron deficient humans or other animals. Such therapeutic compositions are preferably supplied in a three times daily, 21-day calendar pack for monthly iron supplementation therapy. In such a case, absorbed iron could provide approximately 3.0 g per month of iron for hemoglobin regeneration and iron store repletion. As with all the nutritional or dietary supplement compositions of the present invention, it is preferable that the iron supplementation be discontinued for at least a week following administration of the 21-day pack to allow iron absorption rates to remain at their peak during administration weeks.

A further preferred nutritional or dietary supplement composition of the present invention is provided for humans or other animals having iron deficiency anemia. Such a nutritional or dietary supplement composition is beneficial to humans or other animals prior to or during oncology related therapy, pre-dialysis phase of chronic renal failure and repeated blood donations such as for example pre-autologous donations prior to surgery and regular/frequent rare blood-type donors. Additionally, such compositions are suitable replacements for a subset of patients intolerant to intravenous iron. This is especially important for rheumatoid arthritis patients who often become sick when given intravenous iron. It is also important in situations where intravenous iron is contraindicated or not available due to geography, lack of shunt access or intolerance. Suitable compositions of the present invention for treatment of iron deficiency anemia includes 150 mg Ferrochel™ iron, 150 mg succinic acid and 200 mg ascorbic acid, administered up to three times per day for 21 days. As with all the supplement compositions of the present invention, it is preferable that iron supplementation be discontinued for at least a week following the 21 days of iron supplementation administration to allow absorption rates to remain at their peak during administration.

In still another embodiment of the present invention, a nutritional or dietary iron supplement composition is provided for administration in combination with a 28 day course of birth control pills. For example, a commercially available birth control pill comprises about 0.15 mg levonorgestrel and about 30 mcg ethinyl estradiol. A nutritional or dietary iron supplement composition of the present invention comprising about 25 mg Ferrochel™ iron, about 60 mg succinic acid and about 0 to 100 mg ascorbic acid per dosage, can be added with the first 21 days of commercially available birth control pills and omitted from the final 7 days of (placebo) pills. Such a birth control pill components/iron supplement composition may further include folic acid and at least one B complex Vitamin to promote the health of reproductive age women. It is noted that “folic acid” as used herein includes folic acid, folate, folic acid precursors, folate precursors, folic acid derivatives, folate derivatives, folic acid metabolites, folate metabolites and combinations thereof.

As noted briefly above, succinic acid and ascorbic acid promote gastrointestinal iron absorption. Ascorbic acid has been found to enhance gastrointestinal iron absorption only upon oral administration. Gastrointestinal iron absorption is not increased by intravenous administration of ascorbic acid. Succinic acid however, has been found to enhance gastrointestinal iron absorption both upon oral administration and upon intravenous administration. Based on this information, it is reasonable to conclude that the iron absorption promotion effects provided by ascorbic and succinic acid are occurring at different sites and/or through different modes of action. Accordingly, iron absorption promotion effects of ascorbic acid and succinic acid may be additive or even possibly synergistic when used together in an iron supplement composition of the present invention.

The modes of action of succinic acid and ascorbic acid iron absorption promoters are not fully understood. Based on pH considerations, it appears that optimum iron absorption occurs in the proximal duodenal area of the intestine. It has been suggested that succinic acid increases iron absorption by exerting an effect on the basolateral cell membranes of intestinal mucosal cells, thereby increasing transfer of iron already absorbed by small intestine enterocyte cells.

As iron consumed in the diet or through oral supplementation reaches the stomach, it may be bound to dietary substances such as phytates found in various grains. Iron bound to such dietary substances inhibits or decreases iron absorption in the small intestine. The mucosal lining of the small intestine contains fingerlike projections called ‘villi’. The villi are lined by cells that are formed in villi clefts and toward the apices of the villi. Enterocyte cells near the apices of the villi are active absorption sites for iron. Iron absorption is inhibited in the small intestine when iron is bound to dietary substances since bound iron is unavailable for absorption by small intestine enterocyte cells. However, when ascorbic acid is present, the ascorbic acid competitively binds to iron, protecting the iron from phytate binding. Iron is soluble at a low pH. Hence, an additional function of ascorbic acid, through its reducing capabilities, is to keep iron soluble for absorption in the acidic environment of the proximal duodenum.

Once iron is transported from the intestinal lumen into small intestine enterocyte cells, it forms a labile iron pool from which iron is then transported across basolateral membranes and into the blood stream. The extent of the labile iron pool regulates the amount of iron absorbed by small intestine enterocyte cells. As the labile iron pool expands, the amount of iron absorbed by small intestine enterocyte cells and the amount of iron transported across basolateral membranes is reduced.

The principal mechanism by which iron overload and thereby iron toxicity can be prevented, is through a very tightly regulated absorption process in which the small intestine enterocyte cells play a key role. Small intestine enterocyte cells regulate the transport and storage of iron. If iron in the small intestine enterocyte cell's intracellular labile iron pool is not transported across the basolateral membranes, then that untransported iron is lost when the enterocyte cells are sloughed off after several days. This is the chief mechanism by which the body excretes unabsorbed iron.

Only about 5 to 25 percent of ingested iron sulfate, the most commonly used supplemental iron compound, is absorbed. Conventional studies often extrapolated early iron absorption data over long periods of time. However, iron absorption does not remain constant over time. Iron absorption rates, regardless of the iron compound used, with or without promoters, show a marked decrease in absorption after the first 20 days of daily iron supplementation. The conventionally accepted average iron absorption rate of 15 percent appears to be accurate only for iron supplementation days 1 through 20. For days 21 through 30, the average iron absorption rate of a ferrous sulfate supplement dropped to 5.1 percent in published data.

In accordance with the present invention a method is described for administering a nutritional or dietary iron supplement composition to maximize or optimize utilization of said administered iron for clinical benefit. The method of the present invention includes administering an iron supplement composition one or more times a day for one or more days, then discontinuing iron supplementation for one or more days, and then repeating the same, i.e., cyclical administration, to affect an increase in iron absorption in a human or other animal. In one embodiment of the present invention using a method of cyclical administration, the number of days of iron supplementation is the same as the number of days of discontinued iron supplementation. The number of days of iron supplementation can be referred to as the “iron supplementation period” and the number of days of discontinued iron supplementation before again beginning the iron supplementation period can be referred to as the “non-iron supplementation period”. The ratio of the iron supplementation period to the non-iron supplementation period during cyclical administration is preferably 0.03 to 30:1. As noted previously, compositions of the present invention may be used independently to promote and/or maintain iron absorption or used in combination with one or more other compositions used in the treatment of one or more diseases having iron deficiency associated therewith. Accordingly, the administration of such other compositions and/or the non-iron components of compositions of the present invention may be continued during the non-iron supplementation period described herein.

Thus, the present invention provides a method for restoring normal blood-iron concentrations in a human or other animal having below normal blood-iron concentrations utilizing cyclical administration of a nutritional or dietary supplement composition of the present invention. The cyclical administration method of the present invention is capable of achieving blood-iron concentration targets, e.g., RBC generation and iron store repletion, in a shorter period of time than that required using conventional continuous administration regimens. Cyclical administration methods of the present invention reduce the period of time necessary to achieve blood-iron concentration targets by about 10 to about 90 percent, preferably by at least 15 percent, over conventional continuous administration regimens.

The period of time necessary to achieve blood-iron concentration targets is reduced using cyclical administration methods of the present invention through exploitation of the approximately 20 days of high iron absorption experienced upon initiating iron supplementation. Such is exploited in two ways. First, through administration of nutritional or dietary supplement compositions of the present invention, significantly greater amounts of iron are absorbed while minimizing or eliminating unpleasant or harmful side effects. Second, through cyclical administration methods of the present invention, small intestine enterocyte cells are sloughed off within 3 to 7 days during the non-supplementation period thereby clearing the labile iron pool and hence resetting the body's iron absorption mechanism. Cyclical administration methods enhance the rate of iron absorption throughout treatment allowing more iron to be absorbed overall.

Having described the present invention in detail, those skilled in the art will appreciate that modifications may be made to the invention without departing from the spirit and scope thereof. Therefore, it is not intended that the scope of the invention be limited to the specific embodiments described herein. Rather, it is intended only that the appended claims determine the scope of the invention.

1-33. (canceled)

34. A method of administering a composition, the method comprising:
administering said composition, the composition comprising about 50 mg to about 500 mg of one or more forms of iron, about 5 mg to about 500 mg of succinic acid, and about 5 mg to about 500 mg of one or more forms of ascorbic acid, to a human or other animal using cyclical administration.

35. The method of claim 34 wherein said composition is administered at least one time per day using cyclical administration.

36. The method of claim 34 wherein said composition is cyclically administered at least one time per day for a supplementation period of about twenty-one days and a non-iron supplementation period of about seven days.

37. The method of claim 34 wherein said composition is cyclically administered at least one time per day for a supplementation period of about twenty days, and a noniron supplementation period of about ten days.

38. The method of claim 34 wherein said composition is cyclically administered at least one time per day for a supplementation period of about one week, and a non-iron supplementation period of about one week.

39. The method of claim 34 wherein said composition is cyclically administered at least one time per day for a supplementation period and a non-iron supplementation period with the ratio of supplementation period to non-iron supplementation period being approximately 0.03 to 30:1.

40-44. (canceled)

45. A method for treating, preventing, reversing or stabilizing blood-iron concentrations in a human or other animal comprising:
cyclically administering a composition, the composition comprising about 50 mg to about 500 mg of one or more forms of iron, about 5 mg to about 500 mg of succinic acid, and about 5 mg to about 500 mg of one or more forms of ascorbic acid, to a human or other animal for an effective period of time to achieve red blood cell generation and iron store repletion in a time period at least 10 percent shorter than conventional continuous administration.

46. The method of claim 45, wherein the effective period of time to achieve red blood cell generation and iron store repletion is in a time period about 10 percent to about 90 percent shorter than conventional continuous administration.

47-48. (canceled)

49. A method of using a composition comprising:
administering to a human or other animal about 10 mg to about 500 mg of one or more forms of iron; and about 5 mg to about 500 mg of one or more forms of an organic acid, to prevent, stabilize, reverse or treat disorders related to iron deficiency in said human or other animal prior to or during oncology related therapy, pre-dialysis phase of chronic renal failure or repeated blood donations.

50-51. (canceled)

52. The method of claim 34, wherein the composition comprises about 151 mg of said one or more forms of iron; about 150 mg of said one or more forms of an organic acid;
about 200 mg of said one or more forms of ascorbic acid; about 1 mg folic acid; and about 10 mcg Vitamin B₁₂.

53. The method of claim 34, wherein the one or more forms of iron comprises ferrous fumarate iron.

54. The method of claim 34, wherein the one or more forms of iron comprises ferrous fumarate iron and an amino acid chelate of iron.

55. The method of claim 34, wherein said one of more forms of iron include a coating or treatment for controlled release.

56. The method of claim 34, wherein said one or more forms of ascorbic acid are selected from a group consisting of ascorbic acid, salts of ascorbic acid, derivatives of ascorbic acid and compounds having Vitamin C activity.

57. The method of claim 34, wherein said one or more forms of ascorbic acid are selected from the group consisting of ascorbic acid, salts of ascorbic acid, derivatives of ascorbic acid, dehydroascorbic acid, calcium ascorbate, sodium ascorbate, magnesium ascorbate, potassium ascorbate, zinc ascorbate, aldo-lactones, edible salts of aldonic acids, L-threonic acid, L-xylonic acid and L-lyxonic acid.

58. The method of claim 34, further comprising one or more components selected from the group consisting of folic acid and B complex vitamins.

59. The method of claim 34, wherein said one or more forms of iron are selected from the group consisting of bis-glycine chelates of iron.
